(19) United States
(12) Patent Application Publication
(10) Pub. No.: US 2005/0131463 A1
(43) Pub. Date: Jun. 16, 2005

(54) Perfusion chamber for recording evoked and spontaneous electrical activity from submerged acute brain slices

(76) Inventors: Nikolai B. Fedorov, Ho Ho Kus, NJ (US); Timothy H. Michael, Tenafly, NJ (US); Galina Y. Oksman, Fair Lawn, NJ (US); Ana Rosa Chow, Cliffside Park, NJ (US); Gregory M. Rose, Bronx, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/733,817

(22) Filed: Dec. 12, 2003

(51) Int. Cl. A61N 1/00

(52) U.S. Cl. 607/2

(57) ABSTRACT

The invention includes several embodiments of a device for recording electrical activity, particularly the electrical field, from nerve, particularly brain, tissue slices. The device is preferably used for the testing or screening of the effect of physiologically active compounds on the electrical activity of brain tissue slices, which is either electrically evoked or spontaneously occurring. Thus, the invention also includes methods for testing or screening using the device in this manner, for example, to identify compounds that can enhance long term potentiation.

The invention includes several embodiments of a device for recording electrical activity, particularly the electrical field, from biological tissue slices, particularly nerve and brain tissue slices. The device is preferably used for testing or screening for the effect that physiologically active compounds have on brain tissue slices by measuring electrical activity that is either electrically evoked or spontaneously occurring. Thus, the invention is also directed to methods for testing or screening compounds for their effect on electrical activity of biological tissue using the device in this manner. It is particularly useful to identify compounds that can enhance long term potentiation, for example.

### BACKGROUND OF THE INVENTION

The use of in vitro slice preparations of nerve tissue, particularly mammalian brain tissue, for electrophysiological studies is known; see, e.g., R. A. Nicoll et al., J. Neuroscience Methods, vol. 4, pp. 153-156 (1981). For example, hippocampal long-term potentiation (LTP) of synaptic transmission has become a primary experimental model of learning and memory in the vertebrate brain; see, e.g., Matthies et al., J. Neuroscience Methods, vol. 78, pp. 173-179 (1997). LTP has been prominently cast as a key factor in age-related cognitive decline (Foster (1999) Brain Res. Rev. 30:236-249).

Identification of compounds that affect long term potentiation is useful for screening of which compounds may be helpful or harmful to learning and memory. Devices are known for facilitating such screening using nerve tissue, particularly rat hippocampal brain slices. The devices generally include a means for holding the slice in place, a means for immersion or perfusion in a medium, which maintains it physiological state, and a means for electrical stimulation and recording to assess LTP. Compounds to be screened for LTP-affecting activity can be provided in the immersion or perfusion medium, or otherwise delivered to the slice, and their effect on the LTP is assessed. The slices can be tested in the presence of the compound and in the absence of the compound and a comparison made of the duration of long term potentiation in the presence or absence of the compound to determine its effect. In addition to the articles mentioned above, examples of such devices are shown in Haas et al., J. Neurochemical Methods, vol. 1, pp. 323-325 (1979); Shimono et al., J. Neurochemical Methods, vol. 120, pp. 193-202 (2002); and Shepherd et al., Magnetic Resonance in Medicine, vol. 58, pp. 565-569 (2002).

### SUMMARY OF THE INVENTION

The device according to the invention comprises one or more chambers, each chamber having a wall or walls and, preferably, a bottom defining a volume that will hold liquid and having an opening at the top. Each chamber has a pair of rigid electrodes, i.e., a stimulating electrode and a recording electrode, which are provided such that they can be fixed in a position extending into the volume defined by the chamber so that the electrode ends are at a chosen position. In a preferred embodiment, all or part of the bottom of the chamber interior is open through to the bottom of the chamber exterior and the device is provided with a plug that can be inserted through the bottom of the chamber exterior. The plug closes the bottom of the chamber interior so that it holds liquid. In this embodiment, the fixed, rigid electrodes can be provided protruding from the top of the plug. The extent of insertion of the plug into the chamber is variable and a means, e.g., a screw, is provided to lock the plug into its chosen position. In this way, the ends of the electrodes can be adjusted to the desired extent of penetration into the chamber interior by the adjustment of plug position. In this embodiment, the electrode wires can be run to the exterior source through the plug, along the outside of the plug or along grooves provided in the outside of the plug. A sealer material, e.g., wax, can be provided between the plug and the throughhole in which it is inserted to accomplish water tightness.

Each chamber also has a cap with a protrusion, the cap being placeable over all or part of the chamber opening in a defined position such that the protrusion extends a fixed distance into the volume of the chamber. The protrusion is provided so that, when the cap is in its defined position, the protrusion extends into the chamber volume to a defined position which is above the chosen position of the electrode ends. Preferably, the cap and protrusion are provided with a through hole so that the interior of the chamber can be viewed therethrough and air or gas can pass from the chamber interior to the outside therethrough. Thus, in one embodiment, an integral cap/protrusion is preferably hollow and open at the top and bottom such that it provides a rim for setting its position on the top open edge of the chamber and an outline of the protrusion shape extends into the chamber, but the chamber interior is open to the outside through the top. When the device contains multiple chambers, the cap may be provided as a single piece with multiple protrusions for creating a capped volume in each chamber.

The device is also provided with means for holding a tissue sample suspended in the interior volume of the chamber. Preferably this is accomplished by providing two portions (bottom and top) of a material that will hold a tissue sample in place, but which is flexible and allows for perfusion of the liquid in the chamber to the slice, e.g., a net material. The net material also allows for through penetration of the fixed electrode pair, but is sufficiently resilient that such penetration does not hinder the ability of the net material to hold the slice in place even upon multiple uses. The bottom portion of the net material can, for example, be integrated into the chamber horizontally situated above the chamber bottom, removeably attachable into such position in the chamber or situated in such position in the chamber in combination with the brain tissue slice. The top net material can, for example, be an integral or removeably attachable part of the above-described cap with protrusion (particularly stretched across the bottom opening of a hollow protrusion), situated over the brain tissue slice after its placement in the chamber or positioned in the chamber in combination with the brain tissue slice as a sandwich of: bottom net material/slice/top net material. Alternatively, combinations of the above net arrangements can be used. Using any of these arrangements, a sandwich of the net material/slice/net material is ultimately provided in the chamber. When such sandwich is placed in the chamber and the cap is provided in its defined position, the protrusion on the cap pushes the sandwich of net material/slice/net material down to a defined position. The protrusion and position of the electrodes is designed so that, in the final capped position, the bottom net material is pushed down by the movement of the protrusion to its final capped position, which pushes the top net material down onto the sample which, in turn, pushes the bottom net material down. The bottom net material thus flexes down onto the ends of the fixed, rigid and protruding electrodes, which penetrate through the openings of the bottom net material and into the interior of the slice. Because the cap and its protrusion are provided in a defined position and the electrode pair are in a fixed and rigid position, when the thickness of the tissue sample slice, with accompanying net material, is known, the extent of penetration of the electrodes into the slice is known. Thus, multiple tests with different tissue slices of the same thickness can be conducted with reliable results since the device provides the electrodes at a consistent, repeatable distance from each other and at a consistent, repeatable distance of penetration into the slice. The movement of the protrusion to its final capped position may be done manually or in a mechanical or robotized fashion.

The chamber is also preferably provided with a liquid inlet and liquid outlet such that a continuous flow of liquid can be provided. In the preferred use, the chamber(s) are provided with a continuous flow of a liquid for perfusion of brain tissue slice(s). The perfusion can be provided by a tube connecting an external perfusion liquid source to a liquid inlet, e.g., a through hole from the chamber exterior to the chamber interior either through the chamber wall or through the cap/protrusion, and a liquid outlet, e.g., a through hole from the chamber exterior to the chamber interior either through the chamber wall or through the cap/protrusion, connected to a tube for collection of used perfusion liquid. A means for causing flow of the liquid, e.g., a pump in connection with the inlet or outlet tubes, is provided. Preferably, the liquid inlet and liquid outlet are provided at opposing ends of the chamber for optimal perfusion of the tissue sample.

The device is also preferably provided with a light source providing light from a direction beneath the chamber interior. The light illuminates the chamber interior, for example, making the position of the electrodes in the chamber more readily discernible. The light is preferably provided by an LED source, which is useful because it does not generate heat which could damage the tissue sample or alter its properties. In a preferred embodiment, the above-discussed plug for plugging the bottom of the chamber and the light source are integral. For example, the plug is hollow, closed at the top to close the bottom of the chamber interior when inserted and open at the bottom to provide the LED light source therein. The hollow plug is transparent, at least at the top, so that the light shines up through the bottom of the chamber interior. Preferably, the plug comprises one or more concave or flat lenses for directing the light. Particularly preferred is two concave lenses or one concave and one flat lens at the top of the plug for this purpose. The combination of such a light source and a cap/protrusion that is hollow and open at the top and bottom provides the advantage of the operator of the device being able to visualize the tissue sample and aid in its proper placement. Particularly, with the bottom light source, the fixed electrodes produce a shadow when viewed from above, which shadow is even visible when looking through a thin slice of tissue sample. Thus, the tissue sample can more easily be placed such that the electrodes contact it in the proper positions. Because the device is preferably of a small size, the visualizing may be aided by a microscope from above. Other embodiments which provide the light source are possible. For example, at least a bottom portion of the chamber could be of a transparent or translucent material and the light source provided below the chamber (or multiple chambers).

The chambers according to the invention can be constructed of any material that provides the described features, e.g., the material can be shaped to the desired shape and size, it allows for penetration of electrodes through its walls, it does not interfere with electrical activity recordings, it does not damage viability of the tissue, and it does not significantly degrade under the described use conditions. Preferably, it is constructed of a hard plastic material and, preferably, it is transparent to facilitate observation of interior parts. Acrylic plastics are particularly suitable for these purposes, but the material is not limited.

The chamber can be of any shape that allows the described features, e.g., it holds liquid, allows for fixing of electrodes extending into the interior volume, allows for placement of the net material and tissue slice, allows for perfusion of the tissue slice during testing and allows for placement of the cap in a defined position with the protrusion extending into the interior volume to a defined extent. Preferably it has a cubic, rectangular solid or cylindrical shape that is open at the top, closed or closeable at the bottom and, of course, hollow in the interior. The interior volume is of a size and shape to accommodate the discussed elements, e.g., of a cubic, rectangular solid or cylindrical shape which, when capped, is horizontally intersected by the top and bottom nets and contains the protruding electrodes. The bottom is preferably provided with a securing means to hold it in place on a surface, e.g., a vibration-isolation table, so that it is steady but still provides sufficient access to the bottom for electrical and/or lighting connections. This can be provided by a peg affixed to and extending down from the bottom of the chamber, which fits into a hole in a vibration-isolation table. In another embodiment, the chambers can be used in connection with a holding rack for multiple chambers. In one embodiment, the chamber has a square cross-section horizontally such that, when a multiple-chamber device is assembled, the individual chambers are easily assembled together and provide the maximum chamber volume in the space provided. Also preferably, the top of the chamber, which is open, provides a horizontally level rim upon which the cap can be reliably placed in a defined position so that the protrusion of the cap extends into the interior volume to a defined position.

The chambers are preferably small for purposes of economy and, in multi-chamber devices, in order to provide more chambers in a limited space. The chamber's interior volume need only be large enough to provide the structures described herein, hold the tissue sample and allow adequate perfusion thereof. Preferably, each chamber has an external profile, horizontally, of 300-5000 mm², e.g., particularly a circular shape or square shape of 20-30 mm diameter. The profile preferably remains constant along the height of the chamber and the chamber preferably has a height of 20-100 mm, more preferably about 40 to 60 mm. The interior volume of the chamber may be similarly shaped to the exterior but smaller. Thus, the area of a horizontal cross section of the chamber interior volume, uncapped, is preferably about 75-500 mm², e.g., particularly a circular shape or square shape of 10-20 mm diameter. The depth of the interior volume, uncapped is preferably 5-20 mm, more preferably about 6-10 mm. In the above-described embodiment, where the bottom has a through hole with a plug, the depth is adjustable at least for part of the bottom due to the adjustability of the plug. Also, the chamber interior volume can be of varied dimension. For example, the interior volume may have a wider portion at the top for receiving the cap and to facilitate perfusion, but a narrower portion at the bottom for holding the bottom net and receiving the protrusion.

For multi-chamber devices, the chambers can be separately attachable to a surface or can be provided with means for removeably attaching the chambers together so that they hold their position. These means can be, for example, clips, bands around multiple chambers, which press the chambers together, hooks or lips integrated into the chamber which clamp over an adjoining chamber, detachable tape or adhesives, etc. As mentioned above, a multi-chamber device could also be provided by a rack or tray that holds multiple chambers in close proximity to each other. In a preferred embodiment, the multi-chamber devices contain up to 16 chambers, e.g., 4-16 chambers.

The electrodes may be any type of electrode, which meets the requirements of the described device, e.g., be capable of being fixed and rigid, do not significantly degrade under the conditions of use, and provide adequate electrical stimulating and recording properties for electrophysiological studies. The extent of rigidity is relative to the conditions described herein in which they are used. The electrodes need to be rigid enough to maintain position and penetrate into the tissue slice. The tissue slices used here are preferably very thin, e.g., about 200-500 μm thick. Since biological tissue slices are fairly easily penetrated, the electrodes can also be quite thin and still have sufficient rigidity, even upon multiple uses. The electrodes are preferably of a conductive metal wire material that has sufficient strength to maintain rigidity, as discussed, in wire form under the conditions of the invention. The wire electrodes preferably have a thickness of 10-50 μm, particularly about 25 μm. Non-oxidizing platinum/iridium wire electrodes are particularly preferred, which are coated, except for the tips, with a non-stick, durable, insulating material, such as a Teflon. Other useful electrode materials include, but are not limited to platinum and tungsten. It is also preferable if the stimulating electrode is a bipolar electrode, i.e., two wires together. Further, the chamber is also provided with a grounding wire. The grounding wire is run from an external ground into the chamber interior part which will hold the perfusion liquid. Preferably, it is run through the chamber wall into the interior below the bottom net. The grounding wire is preferably of platinum or silver chloride.

The electrodes preferably extend into the chamber interior volume so that they are just below, e.g., about 50-500 μm, more preferably 50-100 μm, below the bottom net when the cap is not in place. They preferably extend from the bottom of the chamber towards the top of the chamber parallel to each other or oriented slightly diagonally toward each other, however, other arrangements are possible. The ends of the stimulating and recording electrodes are preferably spaced apart 20-500 μm, more preferably less than 200 μm and particularly preferably 100 μm±20 μm, from each other. The electrodes extend in the other direction to the exterior of the chamber where connections are made to a suitable electrical stimulating source and recording source in a manner known in the art.

As described above, the electrodes can be provided on a plug, which is inserted through the bottom of the chamber exterior to close the bottom of the chamber interior. But other embodiments are possible such that the chamber interior has fixable electrodes extending into the interior thereof. For example, the chamber can be molded from a plastic around the electrodes such that the electrodes are held in a position extending from the exterior through the chamber bottom into the interior.

The cap and protrusion are preferably an integral piece. For multi-chamber devices, they may have a cap/protrusion for each chamber or a single cap piece having multiple protrusions, one for each chamber. The cap/protrusion can be constructed of any material which provides the described features, e.g., the material can be shaped to the desired shape and size, does not interfere with electrical activity recordings, does not damage viability of the tissue and does not significantly degrade under the described use conditions. For example, it may be of the same materials as the chamber described above. In another alternative, the cap/protrusion can be of another material, e.g., Teflon or metal, such that it can be snugly fit into the opening of the chamber and easily removed. The cap has a top portion with a size and shape such that it is prevented from entering the interior volume of the chamber and a portion below that with a smaller size and shape that corresponds to the size and shape of the inside of the top of the chamber. Thus, the cap can be placed so that the portion below the top enters the interior volume of the chamber in a repeatable, defined position and is stopped in that position by the top portion contacting the top edge(s) of the chamber opening. In this way, the protrusion, which is in a fixed position with relation to the cap, preferably integral with the cap, is also provided in a repeatable, defined position within the chamber interior when the cap is on the chamber. The cap can optionally be provided with a through hole providing gas/liquid communication between the interior of the chamber and the outside environment. This alleviates any problems with pressure differences created in the interior chamber.

The protrusion can be of any design, which fits into the chamber interior and effects positioning of the sample to a defined position, whereby the electrodes penetrate into the interior of the sample. Non-limiting examples of embodiments for achieving this follow. The protrusion preferably has a circular, square or rectangular shape, in horizontal cross-section, and, when the cap is on, extends into the interior an amount such that the above-discussed preferred depth of penetration of the electrodes into the sample is achieved. In a preferred embodiment, the protrusion is hollow, thus, only the outer shape of bottom horizontal cross-section actually effects the pressing down action. In the hollow protrusion embodiment, the outer shape of the bottom horizontal cross-section is of a size that it is larger than and would circumscribe rather than touch the sample. In this embodiment, the pushing down action on the sample is provided by the described top net material, in which, at the same time, the lower net is pushed down by the outer shape (edge) of the bottom horizontal cross section of the protrusion. The outline of the bottom portion of the protrusion pushes down the top net material around the sample (which rests on the bottom net material) and the top net contacts and pushes the sample and bottom net material down onto the electrode ends which penetrate through the bottom net and into the sample interior. The top net material can be provided to effect this embodiment by: affixing the net across at least part of the bottom opening of the hollow portion (i.e., at least the part that will contact the top of the sample) or affixing the net just inside the hollowed portion of the protrusion so that it will contact the top of the sample or affixing the net inside the chamber just above the sample. Preferably, the top net is affixed just inside the bottom opening of the hollow protrusion. This can be accomplished by stretching net material over a metal or plastic ring, for example, a non-corrosive titanium ring, of a diameter corresponding to the protrusion interior and wedging or affixing the ring into the protrusion interior so that the net is affixed across the protrusion bottom opening. When the protrusion is hollow, it can be provided with openings, e.g., perforations or slots, on its walls to allow fluid flow therethrough to enhance perfusion. In an alternative embodiment, the protrusion is solid throughout its vertical cross-section and the bottom of the protrusion simply pushes the sample, resting in the bottom net, down onto the electrodes. In this embodiment, no top net material is required. This embodiment, however, is not preferred since the top of the tissue sample would have hindered perfusion; see the discussion of perfusion below. In another alternative embodiment, the electrodes may be fixed on or in the protrusion such that they penetrate the tissue sample from the top.

The top and bottom net material can be constructed of any material that provides the described features, e.g., flexibility of the bottom net to allow pushing down of the protrusion onto the sample such that the ends of the electrodes penetrate into the sample interior, strength to hold the sample in place under the pushing conditions, resiliency and/or permeability such the electrodes can pass through and liquid/gas permeability to allow perfusion of the tissue sample. Also, it should not damage viability of the tissue, not interfere with electrical activity stimulation and recording, and not significantly degrade under the described use conditions. The material need not actually be a net as long as the material provides the described properties. In a preferred embodiment, the top and bottom net material is provided by a polymer mesh material, particularly polypropylene mesh material or nylon mesh material, most preferably nylon stocking material. The mesh is preferably 200-500 μm, more preferably about 200 μm.

The placement of the top net material is discussed above. The bottom net material is placed to hold the sample just above the ends of the electrodes. This can be effected by providing a metal or plastic ring of diameter corresponding to the interior of the chamber, stretching net material over the ring and wedging in or affixing the ring into the chamber bottom so that the net is held horizontally across the chamber interior at the desire distance above the bottom. Alternatively, the net material can be otherwise affixed at two opposing ends of the interior chamber. The net need not cover the entire chamber cross section but at least extend over sufficient area to hold the sample above the electrode ends. Alternately, a sandwich assembly of the top net/tissue sample/bottom net can be first provided and this sandwich assembly attached across the chamber interior in any of the above-described ways. For any of the above embodiments of top or bottom net material, the attachment of the net material to the chamber walls or protrusion walls can also be effected by hooks and/or adhesives, etc.

The bottom net is positioned in the chamber interior so that there is room to push the bottom net down onto the electrodes for penetrating the sample while maintaining room below the sample for perfusion thereof. Thus, the bottom net is preferably about 1-10 mm above the bottom of the chamber interior. The protrusion is preferably provided so that it pushes the bottom net (and sample thereon) down at least 50 μm and up to 350 μm, preferably about 100 μm, to effect penetration of the electrodes to a desired distance into the sample interior. For brain tissue samples, the slices are generally 200-500 μm thick, usually about 400 μm thick. In the other dimensions, for example for rats, slices are generally 3-5 mm long and 2-3 mm wide. Because the slicing action typically damages the tissue slices to depth of 50 μm on each side, the device is preferably arranged so that the electrode ends penetrate into the interior of the sample at least 50 μm from either sliced surface.

When used for assessing electrical activity of physiological tissue samples, the chamber is perfused with a medium that aids in maintaining the physiological viability of the tissue sample. Such media are well known in the art. An example of a useful media for maintaining brain tissue slices is artificial cerebrospinal fluid (ACSF: 119 mM NaCl, 2.5 mM KCl, 10 mM glucose, 26 mM NaHCO₃, 2.5 mM CaCl₂, 1.25 mM NaH₂PO₄, 1.3 mM MgSO₄). Depending upon experimental conditions, concentrations of the ACSF components may vary slightly. Also, NaH₂PO₄ may be replaced by KH₂PO₄ or HEPES (hydroxyethylpiperazine ethanesulfonate). The media are generally saturated with an oxygen-containing gas, particularly an O₂/CO₂ mixture, more particularly 95% O₂, 5% CO₂ and is heated to 32° C. The perfusion fluid is provided in the chamber interior in an amount sufficient to cover and immerse the tissue sample when the chamber is capped but not overflow the chamber top. It is preferred that the perfusion fluid is provided as a continuous flow through the chamber, as described above. Preferably, the perfusion flow is provided such that it fills the chamber to about 4 mm above the top of the sample and above the top net material. Static batch filling of the chamber with a perfusion fluid can be used with oxygenation but is less desired. Proper fluid height must take into account any displacement of fluid by the sample, nets and inserted protrusion. It may be preferred to provide the chamber interior with a fluid fill level marking.

The chamber or multi-chamber devices of the invention can be used in any experiment for measuring electrical activity of a small, thin sample of matter, which is penetrable by the electrodes. In particular, the devices are suited for testing of biological tissues which have electrical excitability properties or potential. The devices are particularly suitable for measuring electrical activity of nerve tissue slices, particularly mammalian, amphibian or reptilian tissue samples, especially brain tissue or spinal cord tissue slices. For example, mammalian slices can be from old or young, male or female, rats, mice, guinea pigs or humans. Furthermore, mammalian slices may be derived from mutant mammals, e.g., mutant mice with genetic variations that may be either random genetic variations or directed variations (e.g., PDE4 knock-out mice). In a preferred embodiment, the devices are used and the invention is directed to methods for testing mammalian hippocampal brain tissue slices. Such tissue slices can be provided by known methods. Precise slicers for such biological tissue are known, such as Vibrotome™ slicers, which can be adjusted to differing slice thicknesses. The tissue slices used with the devices of the invention for these methods preferably have a thickness of from 200-500 μm, more preferably about 400 μm. The length and width of the samples is preferably in the range of 5-20 mm. In a preferred embodiment, the device is used to assess long term potentiation (LTP) of a brain slice sample. Particularly, the method involves assessing different compounds for their effect on the LTP of a brain slice sample. Hippocampal LTP of synaptic transmission has become a primary experimental model of learning and memory in the mammalian brain. Rat or mouse hippocampal brain slices have been found to be particularly useful for such model. By comparing the LTP in the hippocampal brain slice, with and without a compound of interest present, the effect of the compound on the LTP, detrimental or beneficial, can be assessed. In this way compounds can be identified which increase LTP as being potentially useful in enhancing learning and memory in mammals, particularly humans. Compounds which decrease LTP can be identified as potentially harmful to learning and memory. While LTP tests have long been known in the art for this purpose, the device according to the invention provides several significant advantages in such testing, as discussed herein, for example.

For testing the effect of a compound on the electrical activity of a tissue, the compound is administered to the tissue. This can be done in several ways. Preferably, the compound is mixed with the perfusion fluid to a desired concentration. For example, compounds for testing can be provided in a concentration of from 1 nanomolar (nM) to 1000 micromolar (μm), particularly 1 nM to 100 μm, more particularly 100 nM to 10 μm. Compounds which may have use in treating memory disorders as identified by methods of the invention include, but are not limited to, alpha-7 nicotinic receptor agonists and PDE4 inhibitors. Such testing using multiple chambers according to the invention can be particularly advantageous. For example, several chambers can be run in parallel with differing concentrations of a compound for testing and with all other conditions the same, the samples preferably each being from the same rat hippocampus. This gives a useful concentration profile for the effect of the compound on electrophysiological properties, e.g., LTP. Alternatively, the compound tested can be administered to the sample by application directly onto the tissue sample or injection into the tissue sample.

It was known to conduct LTP tests generally using an electrical stimulation of from 4 up to 100 Hz. These frequencies can be used according to this invention. But the invention is more preferably conducted at frequencies of, e.g., at 30-50 Hz, more preferably about 40 Hz (gamma rhythm). While the optimal LTP output is generally observed at about 100 Hz, it was determined that using a lower frequency was advantageous when testing tissues in the presence of a compound to see the effect of the compound on the LTP. When the LTP is optimized, the difference between the LTP without compound treatment compared to the LTP with compound treatment is less evident than when a less optimal, lower, frequency is used. The increase in LTP caused by the compound is more readily discerned when the LTP is not already optimized.

Theta rhythms have long been known to be a major brain frequency displayed by learning rodents. More recently, gamma rhythms have been identified as another frequency typically displayed by animals as they learn tasks. Furthermore, a positive correlation between both theta and gamma oscillations has been uncovered in freely behaving animals. (Bragin, A. et al. (1995) J.Neurosci. 15(1), 47-60; Colgin, L. L. et al. (2001), Society for Neuroscience 2001 Annual Meeting Program, Abstract 372.11; Fisahn, A. et al. (1998) Nature 394, 186-189.)

In a preferred embodiment, the invention is conducted with 40 Hz theta burst tetanization. This is a tetanization protocol consisting of ten bursts of 4 pulses each. Each of the 10 bursts is spaced by a 5 Hz (theta rhythm) frequency and the four pulses contained within each burst are spaced by a 40 Hz (gamma rhythm) frequency. Other tetanizing protocols may also be useful which involve multiple spaced bursts and/or pulses.

More preferably, the stimulation intensity is at least 50% of the maximum response and the distance between electrodes is less than 200 μm.

In addition to the above-described embodiments of the method wherein an electrical stimulation is applied—i.e., an evoked response—the devices of the invention can also be used to test a spontaneous response. In this embodiment, no electrical stimulation is provided by the stimulating electrode; only the recording electrode is used to assess the response spontaneously generated by the tissue sample.

In addition to LTP testing, the devices according to the invention can be used for assessing other aspects of evoked or spontaneous electrical activity of biological tissue samples. For example, tissue samples can be electrically stimulated and any discharge (e.g., of neurotransmitters such as neuropeptides or glutamate) from the tissue into the chamber as a result of the electrical stimulation assessed.

The invention described herein preferably provides certain advantages discussed above and below, but the invention should not be considered to be limited only to embodiments having these advantages.

The invention is advantageous in providing reliable recording in electrophysiological experiments. The device provides rigid, fixed electrodes and fixed positioning of the tissue slice by the cap protrusion and net materials. This increases the stability of the system and improves stability of the recorded signals regardless of the rate of perfusion of the tissue, mechanical shocks or changes in level of perfusion fluid. Also, as indicated above, because the stimulating and recording electrodes are rigid and in a fixed position, the distance between the electrodes remains the same even upon testing of multiple tissue samples. Further, when multiple tissue slices of the same thickness are tested, the rigid, fixed position of the electrodes and defined position of the protrusion results in a consistent, repeatable depth of penetration of the electrode ends into the interior of the tissue slice. This is advantageous for repeatability in making comparative tests.

The fixed, rigid nature of the electrodes according to the invention is also advantageous in eliminating the need for manipulating the electrodes into the proper position. Not only is this a savings in time, but also it avoids the need for expensive micromanipulators which are often necessary to properly position non-fixed or non-rigid electrodes.

The absence of need for electrode manipulation is also advantageous in conserving the space needed for a chamber of the device, i.e., the chamber can be smaller since there is no need to access the inside for electrode manipulation. The small size of a single chamber provides the ability to provide a device having multiple chambers, thus, providing the advantage of higher throughput testing or screening. Many experiments in which the device of the invention would be used, including LTP testing, require that the device be provided on a vibration-isolation table. These tables typically provide a limited surface area for placement of the device, typically 1-2 square meters. According to the invention, the single chambers can be 1 inch in diameter and one inch high, for example. Thus, for example, a device with 16 chambers can be provided on such a vibration-isolation table. In contrast, only two or three conventional recording chambers which require electrode manipulation could be provided on such a table.

The testing chambers of the invention can also advantageously be used in a multi-chamber assembly. In addition to the above-noted advantage of the small chamber size, they are also advantageous in their ease of replacement. For example, a multiple chamber device according to the invention can be provided where the chambers are removeably attached to one another or to a frame for holding them. Because the electrodes are provided in fixed positions in the chambers, when it is necessary to replace a worn or damaged chamber, it is easy to remove one chamber and replace it with another which will have the same dimensions and same placement of connections for the electrodes and perfusion liquid.

The rigid, fixed positions of the electrodes and the defined position of the cap protrusion in placing the slice are also advantageous because they position the slice so that the ends of the electrodes penetrate into the interior of the slice. Measurements of LTP through the interior of the tissue slice are better indicative of the actual LTP of the tissue in vivo because the interior of the slice is less prone to damage by the slicing process or exposure to the environment after slicing. Slicing of brain tissue irreversibly damages the slice surface to a depth of about 50 μm on each surface. Therefore, it is preferred to arrange the electrodes and protrusion cap considering the slice thickness so that the ends of the electrodes penetrate at least 50 μm into the slice interior.

The entire disclosure of all applications, patents and publications, cited above and below are hereby incorporated by reference.

### BRIEF DESCRIPTION OF THE DRAWINGS

FIG. 1 shows four side views of a device in various stages of use according to an embodiment of the invention, as described in Example 1.

FIG. 2 shows two top views of a device according to an embodiment of the invention, as described in Example 1.

FIG. 3 shows the results of the LTP tests (40 Hz×4×10 and 100 Hz×4×10 tetanic stimulation) in Example 2.

FIG. 4 shows the effect of the test compound, rolipram, on 40 Hz LTP of a rat brain sample in Example 3.

### EXAMPLES

In the foregoing and in the following examples, all temperatures are set forth uncorrected in degrees Celsius; and, unless otherwise indicated, all parts and percentages are by weight.

### Example 1

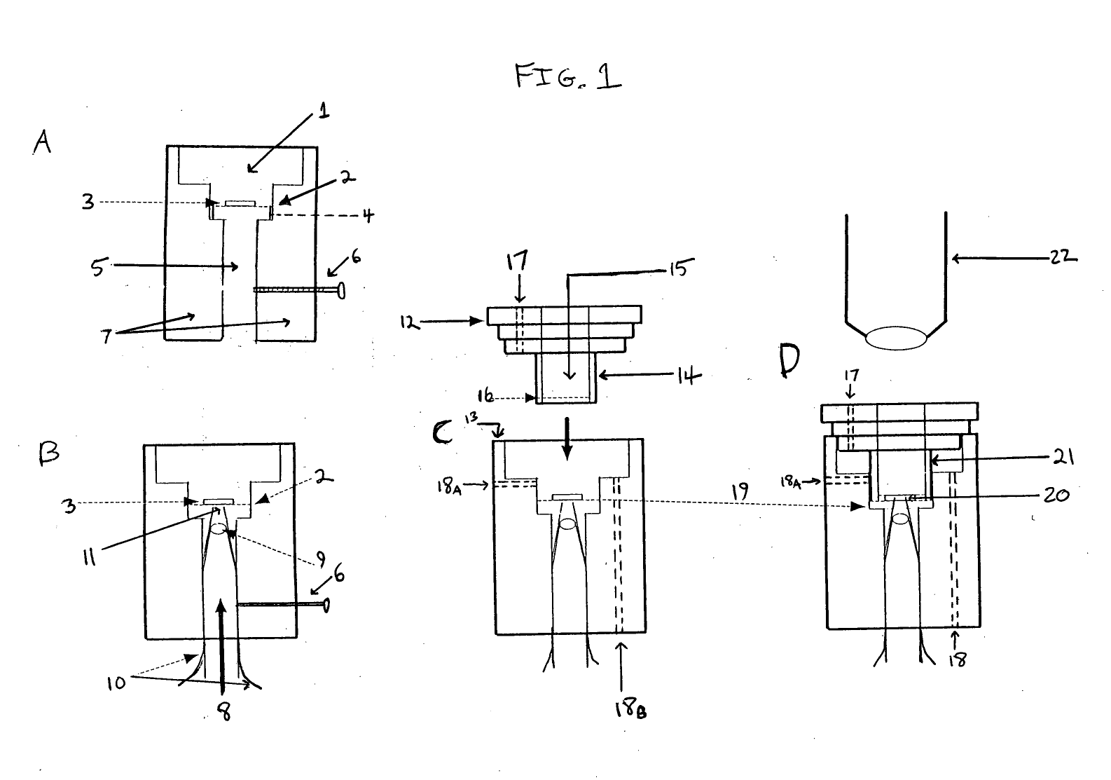

FIG. 1 shows four side views of one embodiment of a device according to the invention in several stages of its arrangement of parts.

Side view A shows a device without the cap/protrusion and without the light/electrode combination. The device has a chamber interior (1) defined by an exterior wall (7). The chamber interior has a bottom net (2) stretched horizontally across its cross section which holds a tissue sample (3). The bottom net is held in position by a ring (4) inserted into the chamber interior. (The ring is not shown in the other drawings so that other elements are more easily seen.) The bottom of the chamber interior is open providing a through hole (5) down to the bottom of the device. The device also has a screw (6) extending through the wall to the through hole for adjustably holding the light source/electrode holder combination, described below, in place.

Side view B shows the above described chamber with a light source/electrode holder combination (8) extending up through the through hole (5). The light source/electrode holder combination is inserted in the direction shown by the arrow. The combination, optionally together with a sealer material, snugly fits the through hole (5) so that the chamber interior will hold liquid. The combination contains or is in communication with a light source to provide light through its top (9). The combination also contains or holds the electrodes (10) so that they extend from external equipment for electrical stimulation and recording (not shown) up through the through hole so that the electrode ends (11) extend into the chamber interior. The electrodes can be adjustably positioned just below the part of the bottom net (2) which holds the sample (3) by locking the combination (8) in place with the screw (6).

Side view C shows a cap/protrusion for use with the chamber. The cap/protrusion has a capping part (12) which has an edge corresponding with the upper edge of the chamber wall (13) to hold the cap/protrusion in a defined position on the chamber. The protrusion (14) is provided to extend down into the chamber interior (see side view D) when the cap/protrusion is in its defined position. The protrusion is hollowed out (15) and open at the top and bottom. The protrusion has a top net (16) horizontally across its bottom opening or just inside its bottom opening. The capping part also has an air release through hole (17) for releasing any excess pressure in the chamber when capping it. The chamber is provided with the through hole from the side (18A) for continuous passage of perfusion liquid into the chamber interior, when capped. The outlet for the perfusion liquid is provided by a through hole (18B) from the chamber interior to outside which outlet/inlet circuit is in communication with a liquid source (not shown) and a pump for liquid flow (not shown).

Side view D shows the cap/protrusion placed in its defined position on the chamber. The protrusion with top net is positioned to contact the top of the sample and push the sample and the bottom net down (arrow 19 showing movement of bottom net with sample) so that the electrode ends penetrate into the sample interior (20). The side walls of the protrusion (21) contain openings (not shown) so that perfusion fluid flowing from the inlet (18A) to the outlet (18B) flows through the hollow portion of the protrusion as well as the chamber interior to achieve perfusion of the sample on all sides, i.e., through the top and bottom nets. Because the protrusion is hollow and open at both ends, the sample can be viewed from the top, preferably by a microscope (22).

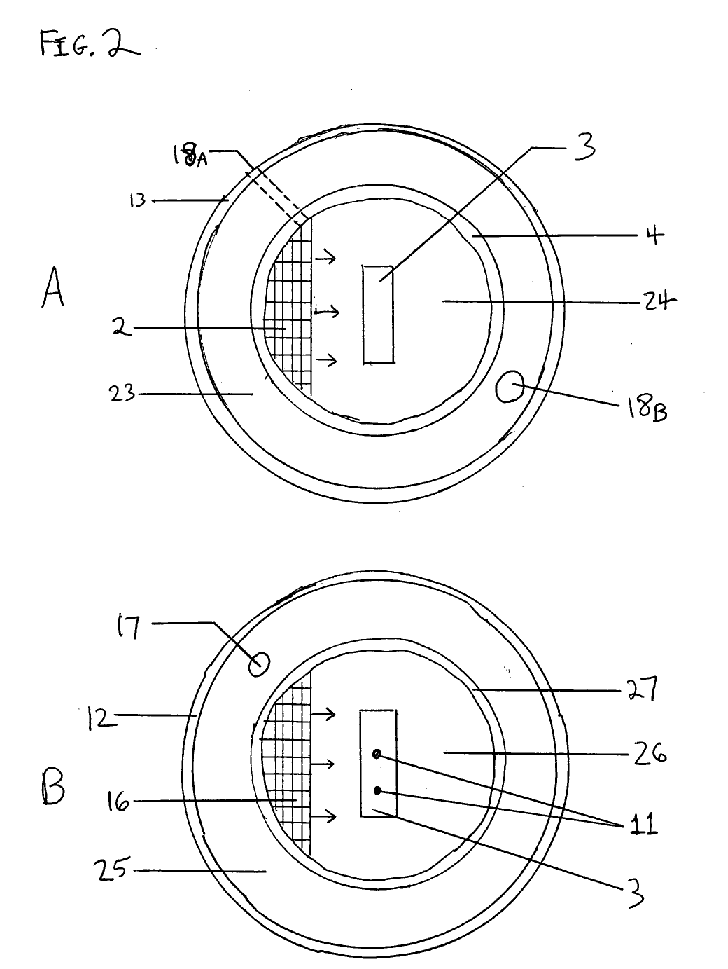

FIG. 2 provides two top views of the device. Top view A shows the chamber without the cap/protrusion. It shows the upper edge of the chamber wall (13), a first part of the chamber interior which is of lesser depth (23) and a second part of the chamber interior which is of greater depth (24). It also shows the ring (4) which holds the bottom net (2). For ease of viewing, the bottom net is shown only partially and it actually extends across the entire portion (24) as shown by the directional arrows. The figure also shows a sample (3) which rests on the bottom net. It also shows the through hole (18B) for perfusion flow outlet. Top view B shows the device with the cap/protrusion in place. The figure shows the cap top (25) with capping part (12), which corresponds to the upper rim of the chamber. It also shows the hollow part of the protrusion open at the top and bottom (26). It also shows a ring (27) which fits inside the protrusion and holds the top net (16). For ease of viewing, the top net is shown only partially and it actually extends across the entire portion (26) as shown by the directional arrows. The drawings are not to scale, but the size of the hollow part, ring and top net is such that it fits inside the second part of the chamber interior (24). The figure also shows a sample (3) which is below the top net. Further, it shows that the position of the electrode ends (11), which are below the sample, are visible as shadows through the sample when viewed from the top. The above-discussed light source provided from below the chamber results in the shadowing by the electrodes. Also shown is the through hole (17) for excess pressure release.

### Example 2

A twelve-chamber assembly of chambers according to the embodiment of the invention described in Example 1 is prepared for measurement of twelve slices simultaneously. Each chamber is prepared such that the top ends of the electrodes are placed 50 μm below the bottom net. Bipolar stimulation is utilized and the distance between electrodes is set to 100 μm. Such an assembly with fixed electrodes may be utilized for numerous experiments without readjustment of the electrodes. Individual chambers in the assembly may be replaced as necessary with new chambers.

Electrophysiology experiments are performed on a 6-month old male rat. 400 μm hippocampal slices from the rat are prepared using a VIBROTOME™ (vibroslicer WPI; World Precision Instruments) to assure precise thickness. Slices were incubated for 2 hours at room temperature in a perfusion solution containing, in mM, 119 NaCl, 2.5 KCl, 2.5 CaCl₂, 1.3 MgSO₄, 1.25 NaH₂PO₄, 26.0 NaHCO₃, 10 glucose and equilibrated with 95% O₂ and 5% CO₂ (pH 7.3-7.4) at room temperature. Slices are placed on the bottom net of each chamber and constantly perfused with this perfusion solution at 32° C. The perfusion rate is set at 1 ml/min and maintained with a peristaltic pump (Dynamax, Rainin).

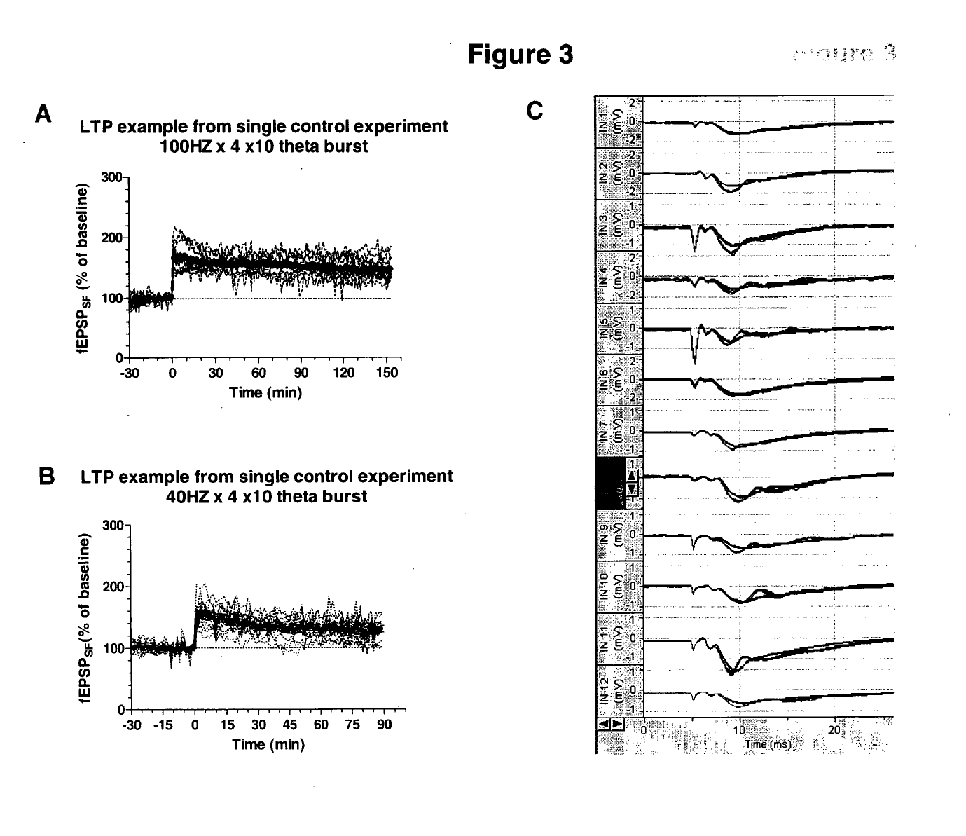

A cap with hollow protrusion and top net is lowered by 100 μm using visual guidance aided by LED light from the bottom of the chamber such that the electrodes penetrate 50 μm into the str. radiatum CA1 region of the hippocampus. The system is equilibrated for twenty minutes before starting measurements. Perfusion, as stated above, is continued. Field recordings of excitatory postsynaptic potential (FEPSP) are obtained from str. radiatum CA1 region. Signals are filtered at 1 kHz and digitized at 5 kHz. Simultaneous recording from the twelve slices is measured using electrophysiological software, Pclamp 9.0 (Axon Instruments), and a A/D board (Axon Instruments). Baseline was obtained every minute using a single pulse of 100 μsec duration with current from 10-30 μA. After 30 minutes baseline recording, single tetanus of 100 Hz theta burst (100 Hz×4×10) or 40 Hz theta burst (40 Hz×4×10) was applied. 30 seconds after tetanus, baseline was continued to be collected as before. The plot of FIG. 3A represents LTP induced by 100 Hz protocol. Dashed lines show time-course of recording from 12 single chambers with the bold line representing an average plot. Recording parameters throughout 3 hour experiment remained stable. FIG. 3B represents LTP induced by 40 Hz theta burst protocol. Dashed lines show time-course of responses from 12 single chambers with the bold line representing an average plot. LTP induced by relatively low frequency (40 Hz vs. 100 Hz) was smaller, that is 130% at 40 Hz vs. 150% at 100 Hz after 1 hour of recording LTP, LTP₆₀. FIG. 3C shows superposition of 10 single responses taken before tetanus and 10 responses taken 50-60 min following 40 Hz tetanic stimulation for 12 chambers.

### Example 3

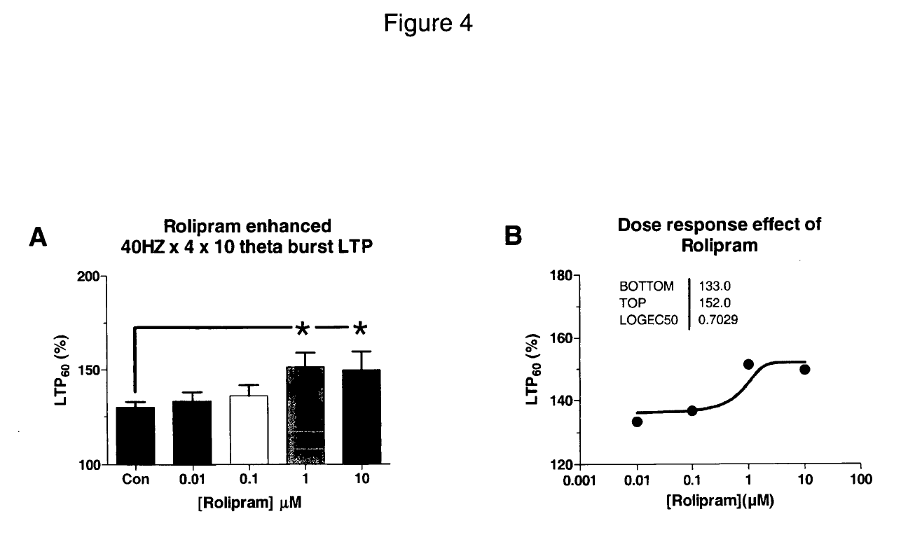

PDE4 inhibitor, rolipram, is tested to determine the effect of the compound on LTP. The twelve chamber assembly as used in Example 2 is used. Baseline is obtained every minute using a single pulse of 100 μsec duration with current from 10-30 μA. After 15 minutes baseline recording, rolipram is added to the perfusion fluid for the twelve chambers in the following concentrations: 0.01 μM for 3 of the chambers, 0.1 μM for 3 other chambers, 1 μM for 3 other chambers and 10 μM for the remaining 3 chambers. After 15 minutes, single tetanus of 40 Hz theta burst (40 Hz×4×10) is applied. 30 seconds after the single tetanus, baseline is continued to be collected as before and LTP was measured 60 minutes after tetanization. The experiment is repeated three more times with the concentrations rotated through the 4 sets of 3 chambers to account for variability between chambers. FIG. 4A shows % of facilitation of the LTP 60 minutes after tetanic stimulation by different concentrations of rolipram. Effect of rolipram at 1 μM and 10 μM was significant (p<0.01, 4 rats, 12 slices for each concentration). FIG. 4B represents the dose-response curve of rolipram. LOGEC 50 values are determined using Prizm4 software (Graphpad).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A device for recording electrical activity of a biological tissue slice which comprises:
a chamber having a wall and a bottom defining a chamber interior volume that will hold liquid and being open at the top with an upper edge defining the opening,
a stimulating electrode wire and a recording electrode wire, which are both rigid and fixable at a position extending into the chamber interior volume so that the electrode ends are at a chosen position within the volume, both electrode wires also extending outside the chamber to electrical stimulating and recording devices, respectively,
a cap with a protrusion, the cap having a capping part corresponding to at least part of upper edge defining the opening of the chamber so that it can be placed over the chamber opening in a defined capped position, the protrusion being part of or connected to the cap such that, when the cap is placed over the chamber opening in its defined position, the protrusion extends a fixed distance into chamber interior volume, and
a liquid-permeable and flexible bottom net material provided across at least part of a horizontal cross section of the chamber interior volume for holding a biological tissue slice within the chamber interior volume,
wherein the extending of the protrusion a fixed distance into the chamber interior volume and the fixing of the electrode wires so that the electrode ends are at a chosen position within the chamber interior volume are such that, when the cap is in its defined capped position and the bottom net material is holding a biological tissue slice, the movement of the protrusion into the chamber interior volume results in penetration of the electrode ends into the interior of the biological tissue slice.

2. The device of claim 1, wherein:
the cap is open to the exterior above the protrusion,
the protrusion is hollow with openings at the top and bottom such that the interior chamber volume is open to the top through the protrusion and cap,
a liquid-permeable and flexible top net material is provided across at least part of a horizontal cross section of the chamber interior volume above the bottom net material and across at least part of a horizontal cross section of the bottom opening of the protrusion,
wherein, when the cap is in its defined position and the bottom net material is holding a biological tissue slice, the outline of the bottom of the protrusion circumscribes but does not touch the tissue slice, the top net material contacts the top of the biological tissue slice and the movement of the protrusion pushes the top and bottom net materials and the biological tissue slice down onto the ends of the electrodes so that they penetrate through the bottom net material into the interior of the biological tissue slice.

3. The device of claim 2, wherein the top net material is provided across the bottom opening of the protrusion or just inside the bottom opening of the protrusion.

4. The device of claim 1, wherein:
the bottom of the chamber interior volume has a bottom through hole extending down to the exterior,
the device further comprises a plug which can be inserted into the bottom through hole such that, optionally with the use of a sealer material, the plug provides at least part of the bottom of the chamber interior volume so that it will hold liquid,
the plug is adjustable as to its extent of insertion into the bottom through hole and a means for holding the plug in a selected position is provided, and
the plug holds the electrode wires so that they extend from outside the chamber, through the bottom through hole and into the chamber interior volume to the their fixable position extending upward into the chamber interior which is adjustable by adjusting the extent of insertion of the plug.

5. The device of claim 4, wherein the means for holding the plug in a selected position is provided by a screw threaded through the chamber wall and into the bottom through hole to contact and hold in place the plug inserted therein.

6. The device of claim 4, wherein the electrode wires are attached to the outside wall of the plug, the electrode wires run inside the plug and out the top or the electrode wires run inside a slot in the outside wall of the plug.

7. The device of claim 2, wherein:
the bottom of the chamber interior volume has a bottom through hole extending down to the exterior,
the device further comprises a plug which can be inserted into the bottom through hole such that, optionally with the use of a sealer material, the plug provides at least part of the bottom of the chamber interior volume so that it will hold liquid,
the plug is adjustable as to its extent of insertion into the bottom through hole and a means for holding the plug in a selected position is provided, and
the plug holds the electrode wires so that they extend from outside the chamber, through the bottom through hole and into the chamber interior volume to the their fixable position extending upward into the chamber interior which is adjustable by adjusting the extent of insertion of the plug.

8. The device of claim 7, wherein the means for holding the plug in a selected position is provided by a screw threaded through the chamber wall and into the bottom through hole to contact and hold in place the plug inserted therein.

9. The device of claim 7, wherein the electrode wires are attached to the outside wall of the plug, the electrode wires run inside the plug and out the top or the electrode wires run inside a slot in the outside wall of the plug.

10. The device of claim 2, which additionally comprises a light source providing light directed up from below and into the chamber interior volume such that, when the device is capped and a biological tissue slice is contained, shadows of the electrode wire ends can be seen through the biological tissue slice when viewed from above through the open cap and protrusion.

11. The device of claim 7, which additionally comprises a light source providing light directed up from the plug, which is transparent at the top, and into the chamber interior volume such that, when the device is capped and a biological tissue slice is contained, shadows of the electrode wire ends can be seen through the biological tissue slice when viewed from above through the open cap and protrusion.

12. The device of claim 11, wherein the light is provided by an LED within the plug and the plug is transparent at the top.

13. The device of claim 11, wherein the plug comprises one or more concave or flat lenses at the top for directing the light.

14. The device of claim 1, which further comprises an inlet and outlet to the chamber interior volume for continuous flow of a perfusion liquid therethrough.

15. The device of claim 2, which further comprises an inlet and outlet to the chamber interior volume for continuous flow of a perfusion liquid therethrough and the protrusion contains openings in its walls to allow perfusion into the interior of the hollow protrusion.

16. The device of claim 1, wherein the chamber interior volume has a top portion of larger horizontal cross section which receives the cap and a bottom portion of smaller horizontal cross section which contains the bottom net material and receives the protrusion.

17. The device of claim 16, wherein the chamber exterior, the top and bottom portions of the chamber interior volume and the cap and protrusion all have circular horizontal cross sections.

18. The device of claim 16, wherein the chamber exterior, the top and bottom portions of the chamber interior volume and the cap and protrusion all have square horizontal cross sections.

19. The device of claim 1, wherein:
the chamber has an external profile, horizontally, of 300-5000 mm², the profile is essentially constant along the height of the chamber and the chamber has a height of 20-100 mm, and
the interior volume of the chamber has a horizontal cross section of about 75-500 mm² and a depth of about 5-20 mm.

20. The device of claim 1, wherein the electrode wires are of a conductive metal wire material with a thickness of 10-50 μm.

21. The device of claim 20, wherein the electrode wires are non-oxidizing platinum, platinum/iridium or tungsten wire electrodes, partially coated with a non-stick material.

22. The device of claim 1, wherein the electrodes are fixable in a position about 50-500 μm below the bottom net when the cap is not in place, the electrode wires extend from the bottom of the chamber interior towards the top of the chamber parallel to each other or slightly diagonally toward each other, and the ends of the stimulating and recording electrode wires are spaced apart from 20-500 μm from each other.

23. The device of claim 1, wherein the cap and protrusion are an integral piece.

24. The device of claim 2, wherein the protrusion has a circular horizontal cross section and the top net material is provided in the bottom of the protrusion opening by a net material stretched over a metal or plastic ring of a diameter corresponding to the protrusion interior which ring is wedged or affixed into the protrusion interior.

25. The device of claim 1, wherein the bottom of the chamber interior volume has a circular horizontal cross section and the bottom net material is provided across the chamber interior volume by a net material stretched over a metal or plastic ring of a diameter corresponding to the bottom of the chamber interior volume which ring is wedged or affixed into the bottom of the chamber interior volume.

26. The device of claim 2, wherein the top and bottom net material are of a polymer mesh material.

27. The device of claim 1, wherein the bottom net material is about 1-10 mm above the bottom of the chamber interior.

28. The device of claim 1, wherein the protrusion is provided so that, when in capped position, the protrusion pushes the bottom net down at least 50 μm and up to 350 μm.

29. A system for recording electrical activity of multiple biological tissue slices which comprises two or more devices according to claim 1 provided together on a vibration/isolation table.

30. A system for recording electrical activity of multiple biological tissue slices which comprises ten to twenty devices according to claim 1 provided together on a vibration/isolation table.

31. A system for recording electrical activity of multiple biological tissue slices which comprises sixteen devices according to claim 1 provided together on a vibration/isolation table.

32. A method for measuring the electrical excitability properties or potential of a small, thin sample of biological tissue using the device of claim 1, which comprises:
placing the sample on the bottom net material positioned above the top ends of the stimulating electrode wire and recording electrode wire,
providing the chamber interior volume with a perfusion liquid either before or after placing the sample,
placing the cap with protrusion in its defined capped position such that the movement of the protrusion into the chamber interior volume pushes the bottom net material and biological tissue slice down onto the ends of the electrode wires so that they penetrate through the bottom net material into the interior of the biological tissue slice,
optionally, providing a stimulating electrical activity through the stimulating electrode wire, and
recording the electrical activity of the sample through the recording electrode wire.

33. The method of claim 32, wherein the biological tissue is brain tissue or spinal cord tissue slices.

34. The method of claim 32, wherein the biological tissue is mammalian hippocampal brain tissue slices.

35. The method of claim 32, wherein the biological tissue is a tissue slice having a thickness of from 200-500 μm.

36. The method of claim 32, wherein the method records long term potentiation (LTP) of a brain slice sample.

37. The method of claim 32, which further comprises contacting the biological tissue with a test compound before and/or during recording of electrical activity to assess the effect of the test compound on the electrical activity of the biological tissue.

38. The method of claim 37, wherein the method records long term potentiation (LTP) of a brain slice sample.

39. The method of claim 38, wherein the brain slice sample is a rat hippocampal brain slice.

40. The method of claim 38, wherein the test compound is mixed with the perfusion fluid for contacting the sample.

41. The method of claim 40, wherein the test compound is provided at a concentration of from 1 nanomolar (nM) to 50 micromolar (μm).

42. The method of claim 40, wherein multiple, essentially identical chambers, are used to conduct parallel runs of samples at differing concentrations of test compound.

43. The method of claim 38, wherein the LTP is assessed of a sample without test compound and with test compound to assess the effect of the test compound on the LTP.

44. The method of claim 32, wherein a stimulating activity through the stimulating electrode wire is provided as about a 40 Hz theta burst tetanization.

45. The method of claim 38, wherein a stimulating activity through the stimulating electrode wire is provided as about a 40 Hz theta burst tetanization.

46. The method of claim 32, wherein a stimulating electrical activity of at least 50% of the maximum response is provided through the stimulating electrode wire.

47. The method of claim 38, wherein a stimulating electrical activity of at least 50% of the maximum response is provided through the stimulating electrode wire.

48. The method of claim 32, wherein the distance between the stimulating electrode wire end and the recording electrode wire end is less than 200 μm.

49. The method of claim 32, wherein stimulating electrical activity under a tetanizing protocol is provided through the stimulating electrode wire.
